(19) United States
(12) Patent Application Publication
(10) Pub. No.: US 2014/0187845 A1
(43) Pub. Date: Jul. 3, 2014

(54) RADIO-IMMUNO-MODULATION TREATMENT FOR ADVANCED CANCER AND MONITORING TOLERANCE AND CELLULAR IMMUNE RESPONSE AT THE TUMOR SITE

(71) Applicant: Razvan Diaconescu, Montreal (CA)

(72) Inventor: Razvan Diaconescu, Montreal (CA)

(73) Assignee: Razvan Diaconescu, Montreal (CA)

(21) Appl. No.: 14/187,266

(22) Filed: Feb. 22, 2014

Related U.S. Application Data
(63) Continuation-in-part of application No. 13/784,252, filed on Mar. 4, 2013.
(60) Provisional application No. 61/617,565, filed on Mar. 29, 2012.

(51) Int. Cl. A61K 35/14; A61N 5/10

(52) U.S. Cl. CPC A61K 35/14; A61N 5/10
USPC 600/1

(57) ABSTRACT

A combination therapy for treating advanced cancer, comprises, first, performing targeted low dose radiation therapy on a recipient tumoral site to generate an inflammation zone and an immuno-stimulant effect, including release of cytokines and chemokines. Secondly, hematopoietic cells from a suitable donor are administered intravenously in order to initiate an allogeneic reaction. These cells could be collected either as PBMC from an adult donor, or from an UCB donation. The post-radiotherapy inflammation zone will attract the newly injected donor cells to the tumor bed, triggering an immunological cancer cell rejection. The cellular response in the recipient is monitored and post-treatment evaluation for recipients' side effects is also provided.

### RELATED APPLICATIONS

This patent application is a continuation in part of U.S. patent application Ser. No. 13/784,252, filed Mar. 4, 2013, which claims priority from U.S. Provisional Application No. 61/617,565, filed Mar. 29, 2012, the contents of which are hereby incorporated by reference.

### FIELD OF THE INVENTION

This specification proposes a Radio-Immuno-Modulation (RIM) treatment for patients with advanced cancer, a method of evaluating patients' tolerance to the treatment and the cellular immune responses at the tumor site.

### BACKGROUND

Advanced lung cancer is a disease driven by numerous oncogenic mutations, which are very heterogeneous between patients, as well as between each individual patient's primary tumor and metastases. These cancers are refractory to chemotherapy either from the beginning or after a limited period of response to the treatment.

Some current treatment approaches are based on stimulating the patient's immune system. The involvement of the immune system in the anti-tumor response has long been suspected and finally shown in more recent works. For example, lung cancer vaccines use the immune system to generate an anti-tumor response.

A first category of vaccines target the tumor associated antigens (TAA), like melanoma associated antigen (MAGE)-A3, Mucin-1, or epidermal growth factor (EGF). A second category of vaccines are prepared from tumor cells, like granulocyte macrophage colony stimulating factor, gene engineered tumor vaccine (GVAX), or Lucanix. The prime components of such vaccines are the TAA, which the immune system is supposed to target.

Another result that a vaccine must induce is that of a co-stimulatory effect on the responder T cells. This is achieved by either adding an immuno-adjuvant product to the first category of vaccines, or by genetically modifying the lung cancer cells in the second category of vaccines, in order to generate immune stimulating cytokines. Tumor responses have been noted after vaccination. However, lung cancer cells have several ways to turn down such an immune response in the tumor microenvironment. These include a low expression of human leukocyte antigen (HLA) molecules, and the presence of local regulatory T cells associated with immunosuppressive cytokines, i.e. transforming growth factor (TGF)-beta, interleukin (IL)-10, or cytotoxic T lymphocyte antigen (CTLA)-4.

Another way of stimulating the immune system is through ionizing radiation, which has a known systemic effect outside of the irradiation field, termed ‘abscopal’, or “off-target” effect. It is known from pre-clinical models, that low, or non-cytolytic doses of radiation produce so-called “danger signals”. Danger signals up-regulate major histocompatibility complex (MHC) molecules presenting TAA on the surface of the tumor cells. This is accompanied by a local secretion of chemokines which enhance the immune cell traffic through the vascular endothelium. The stromal cells situated within the irradiated field contribute to the secretion of inflammatory cytokines that activate the antigen presenting cells (APC). The latter present TAA and stimulate the generation of cytotoxic T cells targeted towards these TAA.

In some cancer treatment trials on animals, low dose radiation has been combined with various forms of immunotherapy like TAA vaccines, CTLA-4 blockade, or adoptive cell therapy. A synergistic effect was observed with all mentioned approaches, producing significantly higher immunologic changes in the tumor microenvironment when radiation was combined with immunotherapy, compared to radiation or immunotherapy alone. For example, the MHC mean fluorescence intensity (MFI) on the tumor cells increased by 100 units and T cells infiltrating the tumor increased their density by 30%. Also, a significantly higher number of animals resolved their tumors with the combined approach compared to either radiation or immunotherapy alone.

This synergy has also been suspected from clinical trials. Butts et al. treated patients with stage III non-small cell lung cancer in a randomized manner with either a Mucin-1 targeted vaccine or with placebo (Lancet Oncol 2014; 15:59). Out of the 806 patients who had received prior concurrent chemotherapy and radiotherapy, those who received the vaccine reached a 50% survival probability at 31 months, compared to only 21 months for the patients who received the placebo, as seen in FIG. 2.

In another trial, Chi et al. treated 12 liver cancer patients with a low dose radiation of 8 Gy to their tumors, followed by an APC injection (J Immunother 2005;28:129). The autologous APC were harvested by leukapheresis and the injection was given intratumorally. An immune response specific to alpha fetoprotein was observed. In this trial, the treatment was well tolerated and tumor regression was noted in 6 patients.

Yet in another trial, Gulley et al. randomized 30 men with hormone resistant prostate cancer in a 2/1 ratio to receive either standard radiotherapy in combination with a prostate specific antigen (PSA) targeted vaccine, or radiotherapy alone for the control group (Clin Cancer Res 2005;11:3353). A T cell response targeted towards PSA was observed on 13 out of 17 evaluable patients in the vaccine group compared to no response in the control group.

Another form of immunotherapy is allogeneic hematopoietic stem cell transplantation (HSCT). This approach has been used for 40 years and relies on the activity of allogeneic (another person's) immune cells, obtained from a healthy donor. Patients with hematologic malignancies that were refractory to chemotherapy were treated with HSCT, in order to obtain the graft-versus-leukemia effect. So far, this immune mediated anti-tumor approach is the only one to offer patients a chance for a long term cure and survival.

Phase I and II studies using allogeneic cellular immunotherapy, with no hematopoietic engraftment, are also known. The patients included in these studied received a total body irradiation dose of 1 Gy, followed by peripheral blood mononuclear cells (PBMC) from an HLA identical family donor. In order to expand eligibility, a subsequent study was done in the haplo-identical setting, as 95% of patients had 3/6 HLA-compatible donors. Those patients who were not heavily pre-treated did not present donor chimerism and the side effects were acceptable.

Depending on their ethnic origin, approximately 40-80% of patients will not have a suitable adult donor of PBMC for hematopoietic transplantation. For these situations, a newer source of cells has been explored, Umbilical Cord Blood (UCB). These cells are obtained through a volunteer donation at the baby's birth, from the placental blood, and stored in cord blood banks.

They have many advantages over stem cells coming from a volunteer donor, such as faster availability, lower incidence of graft-versus-host disease and no risk for the donor. The disadvantages of UCB transplants have mainly derived from their lower numbers of stem cells, relative to an adult recipient. This will not be a concern in our study, as we are not looking for a complete replacement of the patient's hematopoietic system; our hypothesis is that the immunoreactive donor cells will home to the tumoral sites and develop their anti-tumor activity.

### SUMMARY

The present specification describes a therapy for cancer patients conveniently associating the immune adjuvant effect of targeted radiation with infusions of allogeneic hematopoietic cells from a compatible donor. The advantage of using allogeneic cells rather than the patient's own immune system resides in the fact that the patient's immune system has already shown anergy towards the tumor cells through some of the above mentioned mechanisms. This includes the presence of the patient's own regulatory T cells. In the first embodiment, the allogeneic cells are PBMC from a volunteer donor.

According to the method described herein, patients with cancer still progressing after chemotherapy, are treated with radio-immuno-modulation (RIM). No additional chemotherapy regimens or immunosuppressants like in the HSCT need to be administered. Therefore, hematopoietic engraftment or presence of donor cells outside of the tumor bed and/or the regional lymph nodes is not anticipated. Consequently, the complications typically associated with allogeneic HSCT are not expected.

The first stage of the proposed RIM method uses radiotherapy as an immuno-stimulant. A low dose radiation is targeted on one tumor site. The tumor site is one that has not previously received radiation, to promote immunologically activating changes, as described previously. The fastest growing tumor and/or the largest of the metastases are/is chosen for irradiation in order to direct the immune response towards the most aggressive clones.

Optionally, the patient may be administered a non-cytotoxic amount of an immuno-modulator, in a sufficient amount to trigger an immunomodulation activity, while avoiding cytotoxicity. Cyclophosphamide may be used as the immuno-modulator; a proposed amount is preferably in the range of 200 mg/m² to 300 mg/m².

In the second stage of the proposed RIM method, immunotherapy is used to start an allogeneic reaction by injecting the recipient with T-cells from a donor. In the first embodiment, the T-cells are PBMC obtained from a first degree genetic relative of the patient.

These will migrate to the inflammation zone and destroy the tumor. The donor selection is relevant; the donor should present at least 50% HLA compatibility with the recipient and should preferably be related to the recipient. The donor cells may be obtained from a first degree genetic relative of the patient, in relatively good health according to selection criteria described later.

In a preferred embodiment, the allogeneic stem cell source is the UCB cells obtained from a compatible donor.

The evolution of the tumor is then surveyed preferably for six months, as described in detail below.

The methods described here are preferably applicable in cases of advanced or metastatic cancer, where the disease progressed after at least one chemotherapy treatment. In other words, the described methods are applicable preferably for treatment of chemo-resistant cancers. Preferably, the treatment is recommended for chemo-resistant lung cancers.

The low dose radiation generates local secretion of adhesion molecules as well as chemokines, stimulating chemotaxis. Chemotaxis refers to migration of the immune cells through the vascular endothelium towards the irradiated tumor.

On the other hand, the stromal cells in the tumor contribute to the local secretion of inflammatory cytokines and co-stimulation molecules. As such, the APC become activated, and this in turn stimulates production of the cytotoxic T cells directed towards the specific TAA, as illustrated in FIG. 1.

By combining radiotherapy with immunotherapy, a synergetic effect is obtained compared to radiotherapy or donor cells alone. Immune cells from the systemic circulation, especially the donor cytotoxic T cells, migrate to the tumor site, triggering a neoplastic rejection process.

### BRIEF DESCRIPTION OF THE DRAWINGS

FIG. 1 illustrates danger signals following a low dose radiation;

FIG. 2 illustrates an increase in the survival time for stage III lung cancer patients receiving anti-tumor vaccination after concurrent chemo-radiotherapy;

FIG. 3 illustrates a treatment plan for the recipient and donor below and above the time line, according to an embodiment of the RIM method;

FIG. 4 illustrates an example of the immune related response criteria used by the RIM method; and

FIG. 5 illustrates an embodiment of the preconditioning and post treatment schedule according to the RIM method.

### DETAILED DESCRIPTION

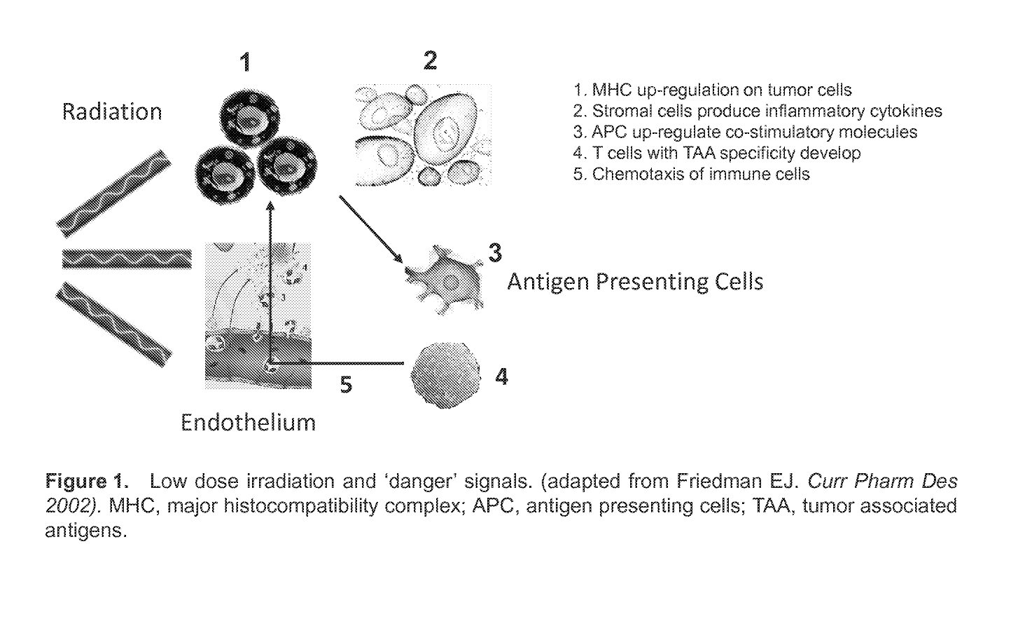

The following detailed description, given by way of example and not intended to limit the present invention solely thereto, will best be appreciated in conjunction with the accompanying drawings FIGS. 1-5.

The terms used in this specification are defined as follows:

“Patient tolerance”: refers to how significant are the post-treatment side effects following RIM; as used herein, this term does not refer to immunologic tolerance;

“Conformal radiation therapy”: is a three dimensional therapy designed to conform the field of radiation to the exact volume of the tissue treated;

“Modulation”: refers to immune modulation representing a change in the direction of an immune reaction towards cancer;

“Human leukocyte antigens (HLA)”: are an important factor in transplantation, while “major histocompatibility antigens (MHC)”, are the equivalent of the HLA in animals;

“Chimerism”: refers to a mixture of patient and donor T cells;

“Abscopal effect”: refers to an immunologic effect of radiation outside of the irradiated zone;

“Performance status”: is a scale which quantifies patients' general well-being and activities of daily life. A performance status of 2 refers to a patient confined to his bed less than 50% of the daytime, and is not considered bedridden;

“Non-cytolytic radiation dose”: refers to a radiation dose insufficient to kill a significant number of tumor cells

FIG. 1 shows the response of a tumor receiving a low dose of radiation. As indicated above, it is known from pre-clinical models, that the danger signals produced after low or non-cytolytic doses of radiation, up-regulate major histocompatibility complex (MHC) molecules presenting tumor associated antigens (TAA) on the surface of the tumor cells, #1 in FIG. 1. The stromal cells situated within the irradiated field contribute to the secretion of inflammatory cytokines, step #2, which, in turn activate the antigen presenting cells (APC), shown at #3 in FIG. 1. The latter present TAA, step #4, and stimulate the generation of cytotoxic T cells targeted towards these TAA. Chemokines are also secreted, and this enhances the immune cell traffic through the vascular endothelium. Cytotoxic T cells migrate to the irradiated tumor, step #5 in FIG. 1.

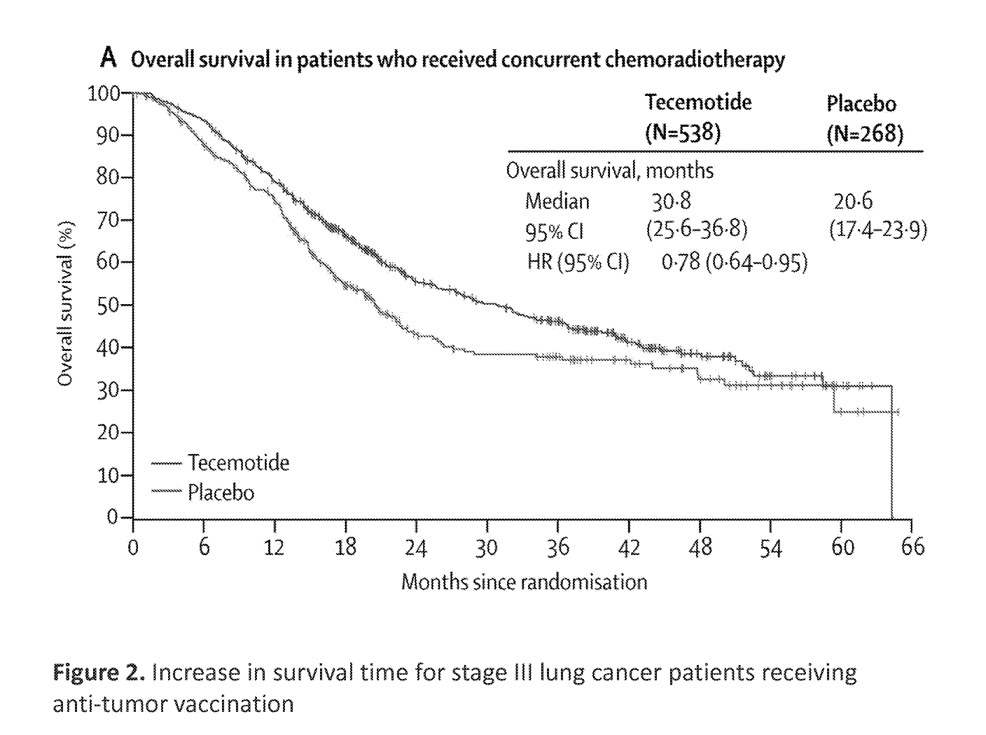

FIG. 2 illustrates an increase in the survival time for stage III lung cancer patients receiving anti-tumor vaccination according to Butts et al. (Lancet Oncol 2014; 15:59). As indicated above, in this study, the patients were treated in a randomized manner with either a Mucin-1 targeted vaccine or with placebo, after having received combination chemo-radiotherapy. Out of the 806 patients who had received concurrent chemo-radiotherapy, median survival was 31 months with the vaccine, compared to only 21 months with the placebo.

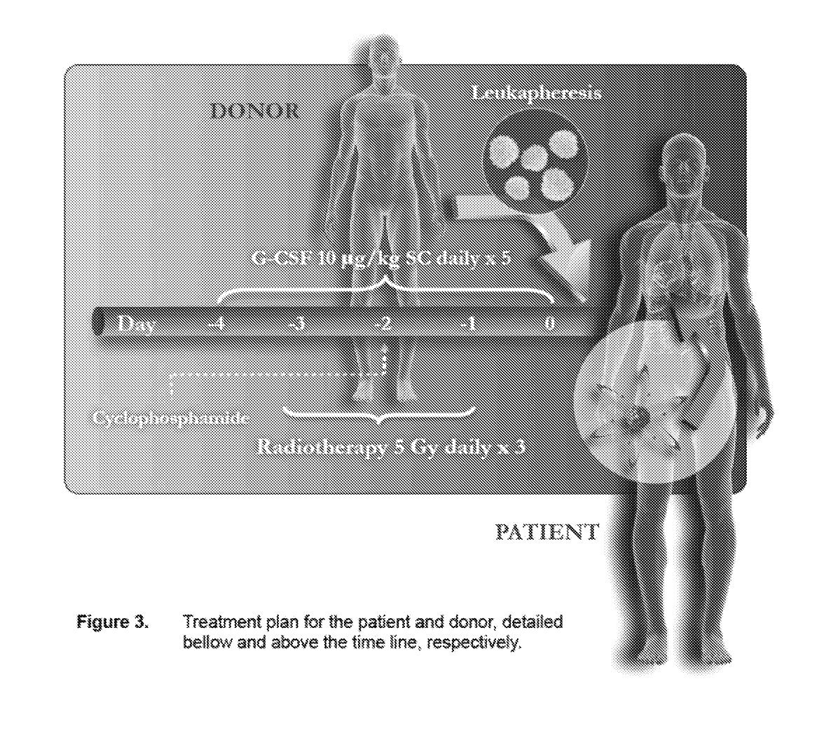

The RIM methods are now described in conjunction with FIGS. 3-5

Pre-Treatment

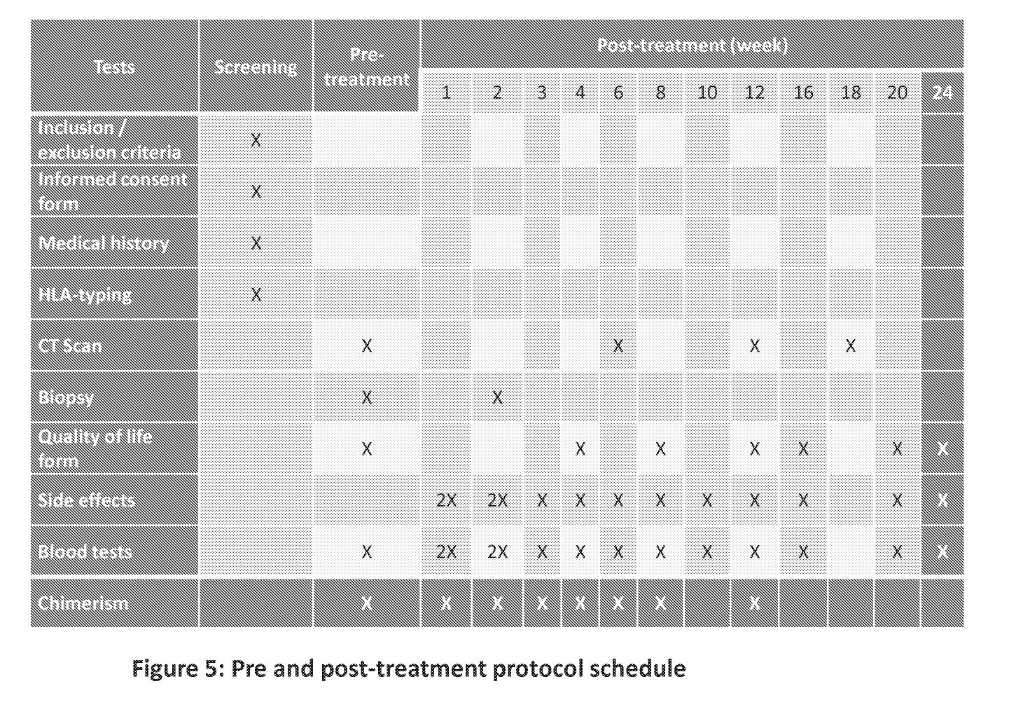

A comprehensive physical examination the patient should be initially performed before commencing the treatment. The patient evaluation is shown in FIG. 5. Patients that are more likely to benefit from this method are selected based on inclusion and exclusion criteria.

Preferably, the patient selection inclusion criteria include:
patients with advanced lung cancer documented by a histo-pathological analysis;
patients who received at least one line of chemotherapy;
patients with least one measurable tumor mass (≧1 cm) not previously irradiated and situated in the lymph nodes, soft tissue, lung, or skeleton;
patients presenting a performance status less or equal to 2 and a life expectancy greater than 3 months;
availability of a compatible allogeneic hematopoietic cell source, either from a PBMC, or UCB donor is also necessary.

Patients for whom the treatment is not recommendable are excluded based on the following exclusion criteria:
Presence of a second active cancer requiring treatment;
Corticosteroid dependency;
Presence of vertebral metastases as the only tumors that could be irradiated;
Decreased diffusion capacity below 40% if a lung tumor was the only metastasis that could be irradiated;
Patients needing urgent radiotherapy.

Patient HLA typing is done for the A, B and DR alleles, as recommended in the setting of allogeneic HSCT.

Donor Evaluation and Selection Criteria

Donor evaluation and selection are described next.

For the embodiment that uses peripheral blood mononuclear cells, the donor should be a first degree genetic relative of the patient, in relatively good health. The donor selection process starts preferably one month prior to the planned treatment, and includes evaluation of the donor health and compatibility.

Donor evaluation includes medical history, physical exam, HLA typing, complete blood count (CBC), electrolytes, glycemia, uric acid, Ca, P, Albumin, lactate dehydrogenase (LDH), liver enzymes, renal, coagulation, blood group, electro cardiogram (EKG) and chest x-ray.

Donor evaluation also includes serology for Human Immunodeficiency Virus (HIV), Hepatitis A, B and C, Herpes Simplex Virus (HSV), Varicella-Zoster Virus (VZV), Epstein-Barr Virus (EBV) and Cyto Megalo Virus (CMV).

Every active medical condition shall be evaluated and stabilised before the PBMC collection, i.e., diabetes, hypertension, chest pain, weight loss, fever, etc.

Aspirin is stopped for one week before and one week after collection, if it is safe to do so. For donors on Aspirin or Plavix needing a central catheter, we favour a femoral line. Donors on anticoagulation medication (Coumadin) who require a central line for collection are switched to low molecular weight heparin.

Donors are considered based on the following donor inclusion criteria: they should be a first degree relative with the patient (parent, sibling or child); have a HLA compatibility of ≧50%; and a performance status of 0 or 1.

Donors are excluded based on the following donor exclusion criteria: a HLA incompatibility in the graft versus host direction (donor homozygous and patient heterozygous for the shared haplotype); a positive serology for HIV, Hepatitis B or C; and an active medical condition that cannot be improved within a month.

For the alternative embodiment when the Umbilical Cord Blood (UCB) cell are used rather than an adult donor (i.e. a first degree genetic relative of the patient), an UCB unit with at least 4 of 6 allele compatibility and at least 2×10⁷ total nucleated cells per kg patient weight should be identified.

Treatment

In the first stage, the patient receives a dose of external radiation of preferably 15 Gy. This dose is divided in three equal fractions of 5 Gy, on Days −3, −2 and −1. Optionally, Cyclophosphamide, 250 mg/m² may be administered on Day −2.

In the second stage of RIM, immunotherapy is used to start an allogeneic reaction by injecting the recipient with a sufficient amount of donor cells. These contain donor T-cells, which are expected to reject the tumor by migration to the inflammation zone.

The day of UCB or PBMC infusion is referred to as ‘Day 0’ and the n^{th} day before that is referred to as ‘Day −n’ (see FIG. 3)

In the embodiment that uses PBMC, donors receive five doses of granulocyte colony stimulating factor (GCSF), each dose calculated as 10 μg/kg, by subcutaneous injection. The GCSF injections are administered from Day −4 to Day 0. PBMC collection is preferably performed through a standard leukapheresis procedure, 2 to 3 hours after the last GCSF injection on Day 0.

Aliquots from the UCB or the PBMC will be stained for flow cytometry with CD34 and CD3 antibodies, in order to measure their respective concentrations. As the optimal cell populations are unknown, these cells will not be selected, but infused entirely; this approach aims to avoid the inadvertent depletion of such cells.

Normally, the patients do not need to receive any pre-medication, unless they have already reacted to previous blood transfusions, necessitating a specific medication. In this case, the same pre-medication is administered prior to PBMC, with the exception of corticosteroids, which should be avoided wherever possible.

PBMC are administered intravenously fresh and unmodified. Donor PBMC should be infused within 24 hours after collection. Preferably, this is performed within 1 hour, in which case the collection bag is kept at room temperature (20-24° C.). If the infusion cannot be performed within 1 hour, the bag is kept in the refrigerator at 4° C. until the infusion.

By combining radiotherapy with immunotherapy, a synergistic effect is obtained, compared to radiotherapy or donor cell administration alone. Immune cell from the systemic circulation, especially the donor cytotoxic T cells, migrate to the tumor site, triggering a neoplastic rejection process.

Post-Treatment

The post-treatment refers to a methodology of monitoring the cellular immune response at the tumor site and the patients' adverse events. Recipients are evaluated preferably during 6 months post-treatment. The patient post-treatment evaluations are shown in FIG. 5.

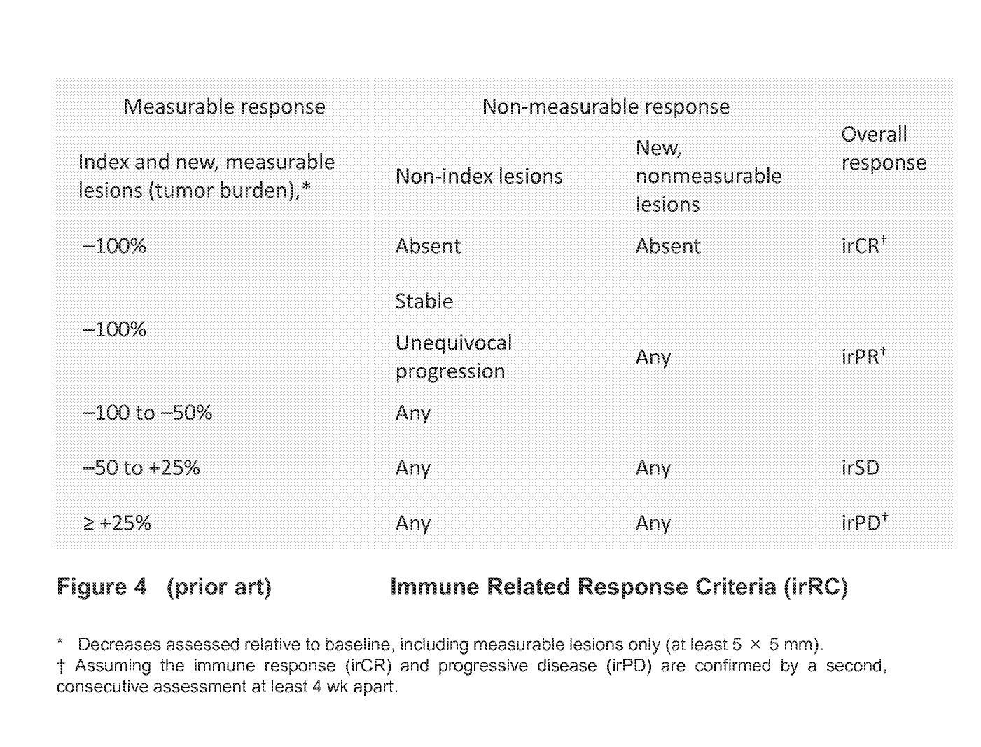

Each patient is evaluated regarding the post-treatment immune condition, the phenotype of the neoplastic cell, and the degree of T cell infiltration. Paired T tests will be used to compare HLA MFI on tumor cells and T cell density between pre and post treatment samples. Tumor response is evaluated using the Immune-Related Response Criteria (irCR) as illustrated in FIG. 4. The survival period without progression is calculated starting from Day 0 until cancer progression, if any. A chimerism analysis is performed for determining the presence of donor cells in the recipient. Adverse events are collected according to the Common Terminology Criteria for Adverse Events (CTCAE) from the National Cancer Institute, version 3.0, and a grade, i.e. mild, moderate, or severe, is attributed to each toxicity. (http://ctep.cancer.qov/protocol Development/electronic applications/docs/ctcaev3.p df).

Treatment tolerance is evaluated by follow-up clinic visits scheduled twice a week for the first 2 weeks, then once a week for the following 2 weeks, then every 2 weeks for 2 months, and every month for the following 3 months, for a total of 6 months, as shown in FIG. 5.

The visits will end either after the 6 months follow-up period, or at the time of disease progression, or whenever the consent of the patient has been waved, if that occurred earlier. Beyond 6 months, standard care will be offered to the patient, according to established clinical guidelines.

The immune response is assessed using tumor biopsies done before and after the treatment for immunological comparisons. The tumor block slides could be stained with CD4 and CD8 antibodies in order to assess the T cell density under the microscope. This will be expressed as T cell numbers on a 400× power field (0.292 mm²), relative to the number of tumoral cells, as well as to the weight of tumoral tissue.

Flow cytometry analysis may be used to determine the presence of immunologic markers like HLA, Fas (CD95) and ICAM-1 (CD54) (tumor cells), CD3, CD4, CD8, CD25 and Foxp3 (T cells). The percentage of the positive cells in these markers as well as their fluorescence intensity will be determined.

The tumor infiltrating T cells are preferably sorted and isolated by fluorescence-activated cell sorting (FACS), from the post treatment biopsy specimen. Blood granulocytes and mononuclear cells are isolated every 2 weeks by density gradient centrifugation. The origins of all these cell populations are determined with polymerase chain reaction (PCR) techniques, quantifying patient and donor specific variable number of tandem repeats (VNTR) bands.

Tumor responses can be assessed through radiologic imaging after 6, 12 and 18 weeks. This is done by CT-scanning and the immune related response criteria are applied, as seen in FIG. 4.

Symptoms attributable to lung cancer are collected monthly according to the FACT/NCCN Lung Symptom Index (FLSI)-12.

### EXAMPLE 1

In this example the radiation is administered to the primary tumor of the recipient. The dose of external radiation is 15 Gy and divided in 3 fractions, on Days −3, −2 and −1.

Cyclophosphamide, 250 mg/m² is administered on Day −2.

Donors receive 5 doses of GCSF, each dose of 10 pg/kg from Day −4 to Day 0. PBMC collection is performed through a standard leukapheresis procedure on Day 0.

The PBMC are administered intravenously, fresh and unmodified, starting at a rate of 40 cc/hour for 15 minutes and increasing with 20 cc/hour every 15 minutes as tolerated, up to a maximum of 100 cc/hour. At the same time, the body temperature, vital signs, and oxygen saturation are monitored every 15 minutes, until a stable flow of perfusion is reached. Thereafter, monitoring is performed every 30 minutes.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

### EXAMPLE 2

This example refers to administering radiation to the largest metastatic site.

The dose of external radiation is 15 Gy and divided in 3 fractions, on Days −3, −2 and −1.

Cyclophosphamide, 250 mg/m² is administered on Day −2.

Donors receive 5 doses of GCSF, each dose of 10 pg/kg from Day −4 to Day 0. PBMC collection is performed through a standard leukapheresis procedure on Day 0. The PBMC are administered intravenously, fresh and unmodified.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

### EXAMPLE 3

This is an example where the radiation is administered to the primary tumor of the recipient. The dose of external radiation is 15 Gy and divided in 3 fractions, on Days −3, −2 and −1.

No Cyclophosphamide is administered in this example.

Donors receive 5 doses of GCSF, each dose of 10 pg/kg from Day −4 to Day 0. PBMC collection is performed through a standard leukapheresis procedure on Day 0. The PBMC are administered intravenously, fresh and unmodified.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

### EXAMPLE 4

This example provides for a method where the radiation is administered to the largest metastatic site. The dose of external radiation is 15 Gy and divided in 3 equal fractions, on Days −3, −2 and −1.

No Cyclophosphamide is administered in this example.

Donors will receive 5 doses of GCSF, each dose of 10 pg/kg from Day −4 to Day 0. PBMC collection is performed through a standard leukapheresis procedure on Day 0.The PBMC are administered intravenously, fresh and unmodified.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

The following Examples 5-8 refer to UCB embodiment

### EXAMPLE 5

In this example the radiation is administered to the primary tumor of the recipient and UCB is used as an allogeneic cell source. The dose of external radiation is 15 Gy and divided in 3 fractions, on Days −3, −2 and −1.

Cyclophosphamide, 250 mg/m² is administered on Day −2.

The UCB unit had been cryopreserved by the cord blood bank. It is thawed and administered intravenously on day 0.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

### EXAMPLE 6

This example refers to administering radiation to the largest metastatic site and using UCB as an allogeneic cell source. The dose of external radiation is 15 Gy and divided in 3 fractions, on Days −3, −2 and −1.

Cyclophosphamide, 250 mg/m² is administered on Day −2.

The UBC unit is thawed and administered intravenously on day 0.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

### EXAMPLE 7

This is an example where the radiation is administered to the primary tumor of the recipient and UCB is used as an allogeneic cell source. The dose of external radiation is 15 Gy and divided in 3 fractions, on Days −3, −2 and −1.

No Cyclophosphamide is administered in this example.

The UCB unit is thawed and administered intravenously on day 0.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

### EXAMPLE 8

This example provides for a method where the radiation is administered to the largest metastatic site and UCB is used as an allogeneic cell source. The dose of external radiation is 15 Gy and divided in 3 equal fractions, on Days −3, −2 and −1.

No Cyclophosphamide is administered in this example.

The UCB unit is thawed and administered intravenously on day 0.

The immune responses are assessed using tumor biopsies done before and after the treatment. Flow cytometry is used to assess the following cell surface markers: HLA, Fas, ICAM-1 on tumor cells, and CD3, CD4, CD8, CD25 and Foxp3 on tumor infiltrating T cells.

The tumor block slides are stained with CD4 and CD8 antibodies in order to assess the T cell density on a 400× magnitude power field.

The tumor infiltrating T cells are isolated by FACS from the post treatment biopsy specimen. Blood granulocytes and mononuclear cells are isolated every 2 weeks with a density gradient centrifugation. The origin of all these cell populations is determined with PCR techniques, quantifying patient and donor specific VNTR bands.

Tumor responses are assessed through radiologic imaging after 6, 12 and 18 weeks. This is done by CT-scanning and the immune related response criteria irCR are applied (see FIG. 4).

1. A method for treating an advanced chemo-resistant cancer, comprising:
generating an inflammation zone at the recipient tumour site using targeted radio therapy; and
injecting intravenously allogeneic cells obtained from a donor.

2. A method as claimed in claim 1, wherein the source of allogeneic cells is Umbilical Cord Blood (UCB).

3. A method as claimed in claim 1, wherein the source of allogeneic cells is peripheral blood mononuclear cells (PMBC).

4. A method as claimed in claim 1, wherein generating an inflammation zone, comprises:
selecting the tumor site to be one of a primary tumor and a large metastatic tumor; and
applying a low dose of daily external radiation targeted at the tumor site, the radiation dose being divided over a number of days prior to the allogeneic cell infusion.

5. A method as claimed in claim 4, wherein the low dose of daily external radiation is 15 Gy and the number of days is three.

6. A method as claimed in claim 1, further comprising optionally administering an immuno-modulator, before injecting the allogeneic cells.

7. A method as claimed in claim 6, wherein the immuno-modulator is Cyclophosphamide.

8. A method as claimed in claim 4, wherein 250 mg/m² of Cyclophosphamide, are administered during the second day of application of the targeted radiotherapy.

9. A method as in claim 1, wherein the tumour is one of a principal lung tumor and a metastatic lung tumor.

10. A method as claimed in claim 1, wherein the tumor is one of a lymph nodes tumor, a soft tissue tumor, a lung tumor and a skeleton tumor.

11. A method as claimed in claim 1, further comprising:
selecting a genetic relative as the donor;
preconditioning the donor to stimulate production of allogeneic cells, by administrating a daily dose of granulocyte colony stimulating factor (GCSF) for a number of preconditioning days; and
collecting the PMBC on the last day of preconditioning.

13. A method for treating an advanced cancer, comprising:
identifying a recipient with advanced cancer;
applying targeted low dose radiation therapy on a recipient tumoral site to generate an inflammation zone and an immuno-stimulant effect including release of cytokines and chemokines;
intravenously administering donor allogeneic cells to the recipient, selected based on a genetic compatibility assessment;
monitoring cellular response in the recipient; and
providing a post-treatment evaluation for recipients' adverse events.

14. A method as in claim 13, wherein the cancer is a chemo-resistant lung cancer.

15. A method as in claim 14, wherein the post-treatment evaluation is a protocol scheduled for 6 months and includes physical examination, CT-scans, biopsy, blood analyses, tests for chimerism, side effects monitoring, and overall quality of life for each recipient.

16. A method as claimed in claim 1, wherein the recipient is identified based on patient inclusion and exclusion criteria.

17. A method as claimed in claim 16, wherein the patient inclusion criteria comprise: patients with advanced lung cancer documented by a histo-pathological analysis; patients who received at least one line of chemotherapy; patients with at least one measurable tumor mass not previously irradiated; or patients presenting a performance status less or equal to 2 and a life expectancy of more than 3 months.

18. A method as claimed in claim 16, wherein the patient inclusion criteria include availability of a family donor with 50% or more HLA compatibility.

19. A method as claimed in claim 13, wherein the donor allogeneic cells comprise PMBC collected from a first degree genetic relative to the recipient.

20. A method as claimed in claim 13, wherein the donor allogeneic cells comprise UCB units with 4 out of 6 HLA compatibility with the recipient and at least 2×10⁷ total nucleated cells per kg patient weight.
